(19) United States
(12) Patent Application Publication
(10) Pub. No.: US 2012/0225837 A1
(43) Pub. Date: Sep. 6, 2012

(54) MEDICINAL AGENT FOR DISEASE ASSOCIATED WITH EPSTEIN-BARR VIRUS, AND METHOD FOR SCREENING OF THE MEDICINAL AGENT

(75) Inventors: Eiichi KODAMA, Kyoto (JP); Masao Matsuoka, Kyoto (JP); Noriyuki Ashida, Choshi (JP)

(73) Assignees: Kyoto University, Kyoto-shi (JP); Yamasa Corporation, Choshi-shi (JP)

(21) Appl. No.: 13/447,727

(22) Filed: Apr. 16, 2012

Related U.S. Application Data
(62) Division of application No. 12/677,116, filed on Mar. 9, 2010.

(30) Foreign Application Priority Data
Sep. 10, 2007 (JP) 2007-233552

(51) Int. Cl. A61K 31/7072; A61P 31/20; A61P 35/00

(52) U.S. Cl. 514/50

(57) ABSTRACT

1-(2-Fluoro-4-thio-β-D-arabinofuranosyl)-5-methyluracil exhibits an anti-EB virus activity, and is therefore effective as a prophylactic or therapeutic agent for a disease associated with an EB virus. Each of a plasmid capable of expressing EB virus-TK and a plasmid capable of expressing human-TK is introduced into a TK-defect cell, thereby producing two types of cells respectively having the plasmids introduced therein. By using the two types of cells, it is possible to screen a medicinal agent which has cytotoxicity against the cell having the plasmid capable of expressing EB virus-TK introduced therein but has no toxicity against the cell having the plasmid capable of expressing human-TK introduced therein. In this manner, it becomes possible to screen a medicinal agent which specifically exhibits an anti-EB virus activity.

### CROSS-REFERENCE TO RELATED APPLICATIONS

The present application is a division of U.S. Ser. No. 12/677,116, filed on Mar. 9, 2010, which is a National Stage (371) of PCT/JP08/66175, filed on Sep. 8, 2008, and claims priority to JP 2007-233552, filed Sep. 10, 2007.

### TECHNICAL FIELD

The present invention relates to an anti-Epstein-Barr virus agent, a medicinal agent for a disease associated with an EB virus, and an effective method for screening of the medicinal agent.

### BACKGROUND ART

An EB virus is considered responsible for various tumors such as B lymphoma, nasopharyngeal cancer and gastric cancer though the incidence rate due to it is not high. As a treatment for such a tumor in an inoperable case, an ordinary anticancer agent is administered, but it is not always an effective treatment. Ganciclovir, an antiviral agent has been reported to be suitable as a therapeutic agent for the tumors (non-patent document 1), but it is not sufficiently effective and hence has not yet been put to practical use.

In addition, a gene therapy for the disease with use of thymidine phosphorylation enzyme (thymidine kinase: TK), which is derived from EB virus related to herpes simplex virus has also been tried in laboratory, but it has not yet been applied to patients.

On the other hand, 1-(2-fluoro-4-thio-β-D-arabinofuranosyl)-5-methyluracil is known as a compound having a low antiviral activity against herpesviruses (non-patent document 2, patent document 1 and patent document 2), but the antiviral activity of the compound against an EB virus and its effect on tumors caused by the EB virus have not been known at all.

Non-patent document 1: Antimicrob. Agents Chemother 2001; 45(7): 2082-91

Non-patent document 2: Antiviral Res. 1998; 39(2): 129-37

Patent document 1: International Publication No. WO91/04982

Patent document 2: JP-A-10-87687

### DISCLOSURE OF THE INVENTION

### Problem to be Solved by the Invention

Therefore, the present invention is intended to find an anti-EB virus agent and a medicinal agent having a marked effect on a disease associated with an EB virus.

### Means for Solving the Problem

No effective screening method for finding a medicinal agent for a disease associated with an EB virus has been reported. Therefore, the present inventors attempted to establish an effective screening method at first and find a medicinal agent having a marked effect on a disease associated with an EB virus by the use of the screening method. As a result, by utilizing TK derived from an EB virus which is present in tumor cells, the present inventors have established a method for screening a medicinal agent which is specifically phosphorylated by the TK to exhibit toxicity against the cells. As a result of screening of various compounds by the above-mentioned screening method, the present inventors have found that 1-(2-fluoro-4-thio-β-D-arabinofuranosyl)-5-methyluracil has an anti-EB virus activity and a marked effect on a disease associated with an EB virus.

Therefore, the present invention has been accomplished on the basis of the above finding and has the following aspects [1] to [4].

[1] An anti-Epstein-Barr (EB) virus agent comprising 1-(2-fluoro-4-thio-β-D-arabinofuranosyl)-5-methyluracil, a salt thereof, or a hydrate or solvate of either of them as an active ingredient.

[2] A prophylactic or therapeutic agent for a disease associated with an EB virus which comprises 1-(2-fluoro-4-thio-β-D-arabinofuranosyl)-5-methyluracil, a salt thereof, or a hydrate or solvate of either of them as an active ingredient.

[3] A prophylactic or therapeutic agent according to the above item [2], wherein the disease associated with an EB virus is a tumor caused by the EB virus or an acute or chronic EB virus infectious disease.

[4] A method for screening a medicinal agent having an anti-EB virus activity which comprises introducing each of a plasmid capable of expressing EB virus-TK gene and a plasmid capable of expressing human-TK gene into a thymidine kinase (TK)-defect cell to produce two types of cells respectively having the plasmids introduced therein; and screening a medicinal agent which has cytotoxicity against the cell having the plasmid capable of expressing EB virus-TK gene introduced therein but has no cytotoxicity against the cell having the plasmid capable of expressing human-TK gene introduced therein, by the use of the above-mentioned two types of cells.

### Advantages of the Invention

The medicinal agent of the present invention has cytotoxicity only against cells in which EB virus-TK gene has been expressed, and it has no cyotoxicity against cells in which human-TK gene has been expressed. Therefore, the medicinal agent may be expected to have anti-EB virus effect, prophylactic and therapeutic effects on a disease associated with an EB virus, and only reduced side effects. Thus, the medicinal agent is useful as a prophylactic or therapeutic agent for a disease caused by an EB virus, such as an acute or chronic EB virus infectious disease or a malignant tumor caused by the EB virus.

In the screening method of the present invention, the utilization of TK derived from an EB virus makes it possible to screen a medicinal agent which is specifically phosphorylated by the TK to exhibit cytotoxicity against cells. Therefore, the screening method is useful as a method for screening a medicinal agent capable of exhibiting anti-EB virus activity.

### BEST MODE FOR CARRYING OUT THE INVENTION

The screening method of the present invention is explained below at first, and then the medicinal agent of the present invention selected by the screening method is explained.

### (1) The Screening Method of the Present Invention

As described above, in the method of the present invention, each of a plasmid capable of expressing EB virus-TK gene and a plasmid capable of expressing human-TK gene is introduced into a TK-defect cell to produce two types of cells respectively having the plasmids introduced therein, and by the use of the two types of cells. A medicinal agent is screened which has cytotoxicity against the cell having the plasmid capable of expressing EB virus-TK gene introduced therein but has no cytotoxicity against the cell having the plasmid capable of expressing human-TK gene introduced therein,

As the TK-defect cell, a TK-defect human tumor cell is preferable. Examples thereof include a TK-defect human osteosarcoma cell (143B). Such a cell is available from ATCC or the like, and the 143B cell mentioned above has been registered as ATCC CRL-8303.

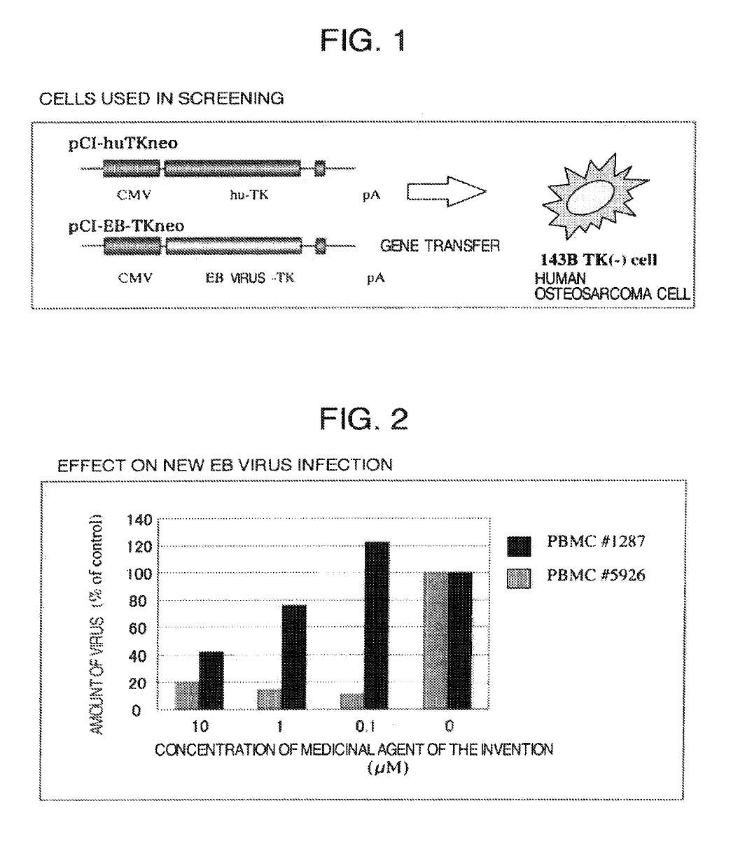

Examples of the plasmid vector capable of expressing EB virus-TK gene or human-TK gene include a plasmid which, as shown in FIG. 1, has suitable antibiotic resistance for selection and a gene coding for EB virus-TK or human-TK which is connected in an expressible state downstream to a promoter.

The gene coding for EB virus-TK or human-TK has already been cloned and its base sequence may be looked up by the use of the data base of National Center for Biotechnology Information (NCBI). Therefore, the gene coding for EB virus-TK or human-TK may be prepared by a conventional method on the basis of the data base information of NCBI.

Next, the TK gene prepared above is connected to a plasmid having suitable antibiotic resistance and, for example, a promoter and a terminator derived from cytomegalovirus (CMV), by a conventional method. Specifically, the gene coding for EB virus-TK or human-TK is connected to a multicloning site downstream to the promoter of pCI-neo Mammalian Expression Vector, a plasmid available from Promega Inc. by a conventional method so as to enable the expression.

The above-mentioned TK-defect cell is transfected with the plasmid prepared. The gene transfection may be carried out by a conventional method (Proc. Natl. Acad. Sci. USA 1987 November; 84(21): 7413-7).

Using the thus prepared two types of cells having the EB virus-TK gene and human-TK gene, respectively, transfected thereto, a medicinal agent is screened which has cytotoxicity against the cell having EB virus-TK gene introduced therein but has no cytotoxicity against the cell having human-TK gene introduced therein, whereby the medicinal agent having an anti-EB virus activity may be screened. In particular, it is possible to express the selective toxicity of a test medicinal agent in terms of a coefficient by measuring a concentration (CC₅₀) of the test medicinal agent at which the cell viability of each of the cells is decreased to 50%, and calculating the ratio between the thus measured concentrations as a selection index (SI).

### (2) The Medicinal Agent of the Present Invention

The active ingredient of the medicinal agent of the present invention is 1-(2-deoxy-2-fluoro-4-thio-β-D-arabinofuranosyl)-5-methyluracil (another name: 1-(2-deoxy-2-fluoro-4-thio-β-D-arabinofuranosyl)thymine). This compound is a well-known compound and may be prepared by a method known in literature or a modification thereof (see non-patent document 2, patent document 1, patent document 2 or the like). This compound may be in the form of a salt, hydrate or solvate. Examples of such a salt include acid adducts such as hydrochloride and sulfate. Examples of the hydrate or solvate include compounds produced by adhesion of 0.1 to 3.0 molecules of water or a solvent to a molecule of the above-mentioned compound or salt thereof. The above-mentioned compound also includes its various isomers such as its tautomers.

The medicinal agent of the present invention is effective as an anti-EB virus agent and a prophylactic or therapeutic agent for a disease associated with an EB virus. Examples of the disease associated with an EB virus include tumors caused by the EB virus and acute or chronic EB virus infectious diseases. Examples of the tumors caused by the EB virus include B lymphoma, nasopharyngeal cancer and gastric cancer.

The dose of the medicinal agent of the present invention is varied depending on the age, body weight, disease of a patient, the seriousness of the disease of the patient, tolerance for a drug, an administration route and the like, and is properly determined by considering these conditions synthetically. The dose is usually chosen in the range of 0.001 to 1000 mg/kg body weight, preferably 0.001 to 100 mg/kg body weight, per day. The medicinal agent is administered in one portion or several portions. As to the administration route, the medicinal agent may be administered by any of oral, non-oral, enteral and topical administrations and the like.

For formulating the above-mentioned compound into a pharmaceutical preparation, conventional additives such as a carrier for formulation, excipient and the like may be used. Examples of the carrier include solid carriers such as lactose, kaolin, sucrose, crystalline cellulose, corn starch, talc, agar, pectin, stearic acid, magnesium stearate, lecithin, sodium chloride, etc.; and liquid carriers such as glycerol, peanut oil, polyvinylpyrrolidones, olive oil, ethanol, benzyl alcohol, propylene glycol, water, etc. The pharmaceutical form may be any form. For example, when the solid carrier is used, examples of the pharmaceutical form include tablets, powders, granules, capsules, suppositories and troches. When the liquid carrier is used, examples of the pharmaceutical form include syrups, emulsions, soft gelatin capsules, creams, gels, pastes, sprays and injections.

### EXAMPLES

The present invention will be concretely explained with reference to a working example, which should not be construed as limiting the scope of the invention.

### EXAMPLE

### (1) Establishment of a Cell Strain

FIG. 1 shows an outline of a method for screening a medicinal agent which is specifically phosphorylated by EB virus-TK to exhibit cytotoxicity against cells.

At first, a primer was designed on the basis of the data base information of NCBI, followed by extracting DNA from B95a cells (J. Virol. 1990; 64(2): 700-5), and the TK gene of an EB virus was amplified by PCR method and cloned in the multicloning site of an expression plasmid vector pCI-neo (pCI-EB-TKneo). Similarly, human-TK gene was also amplified by PCR method and cloned (pCI-huTKneo). Whether the cloned gene fragment was a desired gene or not was judged by determining the base sequence.

Then, TK-defect human osteosarcoma cells (143B) was transfected by lipofection method (Proc. Natl. Acad. Sci. USA 1987 November; 84(21): 7413-7) with the plasmid vector capable of expressing EB virus-TK gene or the plasmid vector capable of expressing human-TK gene, namely, pCI-EB-TKneo or pCI-huTKneo, and cells having each plasmid introduced therein were selected by the use of neomycin.

Using these two types of cells, i.e., the cell having EB virus-TK gene introduced therein (143B/EB virus-TK) and the cell having human-TK gene introduced therein (143B/hu-TK), a medicinal agent was screened which had cytotoxicity against the cell having EB virus-TK gene introduced therein but had no cytotoxicity against the cell having human-TK gene introduced therein.

### (2) Determination of Drug Sensitivity

The drug sensitivity of 143B/EB virus-TK, 143B/hu-TK, 143B TK(−) cells and EB virus persistent infection cells was determined on the basis of the cytotoxicity of each drug. That is, cells (143B/EB virus-TK, 143B/hu-TK and 143B TK(−) cells: 5×10⁴ cells/100 μl; EB virus persistent infection cells: 20×10⁴ cells/100 μl) were added to 96-well flat-bottom culture plates containing 100 μl of various concentrations of each drug. After 5 days of culture, the number of surviving cells was determined by MTT [3-(4,5-dimethylthiazol-2-yl)-2,5-diphenyltetrazolium bromide] assay. The cytotoxicity of the test drugs is expressed in terms of a concentration (CC₅₀) at which the cell viability was decreased to 50%.

### (3) Effect of Drugs

By MTT assay, there was judged whether or not nucleic acid derivatives whose phosphorylation by EB virus-TK has been reported (azidothymidine (AZT), 5-fluoro-2′-deoxyuridine (5FdU), acyclovir (ACV), 1-β-D-arabinofuranosyl-5-bromovinyluracil (BVaraU), ganciclovir (GCV) and 5-bromovinyl-2′-deoxyuridine (BVDU)) had selective toxicity against 143B/EB virus-TK in which EB virus-TK gene had been expressed. In the same manner as above, there were investigated the following nucleic acid derivatives having a structure similar to that of the compound according to the present invention: 1-(2-deoxy-2-fluoro-4-thio-β-D-arabinofuranosyl)uracil (FMAU), 1-(C-cyano-2-deoxy-ribofuranosyl)thymine (1′-CN-dT), β-D-2-methylene-4-thio-β-D-erythropentofuranosyl)thymine (4′-S-DMDT), 1-(2-deoxy-4-thio-β-D-arabinofuranosyl)thymine (4′-S-araT) and 1-(2-deoxy-2-fluoro-4-thio-β-D-arabinofuranosyl)cytosine (4′-S-FMAC).

As a result, as shown in Table 1, no drug having selective toxicity against 143B/EB virus-TK cells could be found except the medicinal agent of the present invention. In addition, as a result of screening of 130 nucleic acid derivatives other than the above-mentioned nucleic acid derivatives in the same manner as above, no drug having a marked effect could be found.

### (4) Effect of the Medicinal Agent of the Present Invention

In the same manner as in the above item (3), the cytotoxicity of the medicinal agent of the present invention against 143B/EB virus-TK cells and 143B/hu-TK (human-TK) cells was investigated by MTT assay to find that as shown in Table 1, the medicinal agent had the highest selection index (SI: more than 2700-fold) for 143B/EB virus-TK cells but had no cytotoxicity against 143B/hu-TK cells even at 300 μM.

TABLE 1
Cytotoxic effect of various drugs

| | CC₅₀(μM) | | |
|---|---|---|---|
| Drug | 143B/EB Virus-TK | SI | 143B/hu-TK |
| AZT | >100 | | >100 |
| 5FdU | 0.40 | 0.7 | 0.27 |
| BvaraU | >200 | | >200 |
| BVDU | 122 | >2.5 | >300 |
| GCV | >100 | | >100 |
| ACV | >100 | | >100 |
| FMAU | 320 | 0.9 | 290 |
| 1′-CN-dT | >100 | | >100 |
| 4′-S-DMDT | >100 | | >100 |
| 4′-S-araT | >100 | | 100 |
| 4′-S-FMAC | 0.066 | 0.6 | 0.040 |
| Medicinal | 0.11 | >2700 | >300 |
| agent of the | | | |
| invention | | | |

### (5) Effect of the Medicinal Agent of the Present Invention on EB Virus Infection Cancer Cells

The cytotoxic effect of the medicinal agent of the present invention on EB virus infection cancer cells such as NC37 cells was investigated by MTT assay. As a result, it was found that as shown in Table 2, the medicinal agent of the present invention was more than 10 times as effective as an existing drug GCV against NC37 cells in which EB virus-TK gene had been expressed. However, neither of them was effective in cells in which EB virus-TK gene had not been expressed.

TABLE 2
Cytotoxic effect on EB virus infection cancer cells

| | Expression | Medicinal agent of | GCV |
|---|---|---|---|
| Cell | of TK | the invention μM | μM |
| B95a | + | 2.2 | 36 |
| NC37 | + | 2.1 | 27 |
| Raji | − | 22 | 55 |
| Namalwa | − | >100 | >100 |
| HS-Sultan | − | >100 | >100 |

### (6) Effect on Acute Infection

B lymphocytes were separated from the blood of a healthy person. The B lymphocytes were infected with an EB virus in the presence of the medicinal agent of the present invention and cultured for 2 weeks. Thereafter, the infected B cells were recovered and then washed, followed by extraction of DNA. The amount of EB virus DNA in the B lymphocytes was determined by real-time PCR and taken as a virus copy number. FIG. 2 shows the results obtained in two typical cases (PBMC#1287 and PBMC#5926). It was proved that as shown in FIG. 2, the medicinal agent of the present invention reduces the amount of EB virus DNA in the B lymphocytes and inhibits new infection.

(7) Effect on Cells with a Chronic Active EB Virus Infectious Disease

A chronic active EB virus infectious disease is an unfavorable infectious disease caused by infection of not B cells but natural killer T cells (NKT cells) with an EB virus. As such a chronic infection cell strain, KAI3 cells (Clin. Exp. Immunol. 1999 March; 115(3): 385-92) were purchased from Health Science Research Resources Bank, and the effect of the medicinal agent of the present invention on them was investigated by MTT assay. GCV was used as a control agent. As a result, it was found that as shown in Table 3, the medicinal agent of the present invention was clearly more effective than GCV.

Thus, it was proved that the medicinal agent of the present invention is more than 10 times as effective as GCV whose effect has been reported. Therefore, it is considered that the medicinal agent of the present invention has cytotoxicity only against cells in which EB virus-TK gene has been expressed, and that the medicinal agent can ablate EB virus-infected cells irrespective of whether the infection is acute or chronic.

TABLE 3
Effect on cells with a chronic active EB virus infectious disease

| | | |
|---|---|---|
| Medicinal agent of the invention | 4.7 | μM |
| GCV | 105 | μM |

### INDUSTRIAL APPLICABILITY

The medicinal agent of the present invention is useful as a prophylactic or therapeutic agent for a disease caused by an EB virus, such as an acute or chronic EB virus infectious disease or a malignant tumor caused by the EB virus, which has only reduced side effects. The screening method of the present invention is useful as a method for screening a medicinal agent capable of exhibiting an anti-EB virus activity specifically.

### BRIEF DESCRIPTION OF THE DRAWINGS

[FIG. 1] A schematic view showing an outline of the screening method of the present invention.

[FIG. 2] A graph showing the effect of the medicinal agent of the present invention on the amount of EB virus DNA in B lymphocytes.

what we claim is:

1. A method of treating a disease associated with an Epstein-Barr (EB) virus in a subject in need thereof which comprises administering to said subject an effective amount of 1-(2-deoxy-2-fluoro-4-thio-β-D-arabinofuranosyl)-5-methyluracil or a salt thereof.

2. The method according to claim 1, wherein the disease associated with an EB virus is a tumor caused by the EB virus.

3. The method according to claim 1, wherein the disease associated with an EB virus is an acute EB virus infectious disease or a chronic EB virus infectious disease.

4. The method according to claim 1, wherein said method comprises administering to said subject an effective amount of 1-(2-deoxy-2-fluoro-4-thio-β-D-arabinofuranosyl)-5-methyluracil.

5. The method according to claim 1, wherein said method comprises administering to said subject an effective amount of a salt of 1-(2-deoxy-2-fluoro-4-thio-β-D-arabinofuranosyl)-5-methyluracil.

6. The method according to claim 1, wherein the disease associated with an EB virus is a tumor selected from the group consisting of B lymphoma, nasopharyngeal cancer and gastric cancer.

7. The method according to claim 1, wherein said effective amount ranges from 0.001 to 1000 mg/kg body weight per day.

8. The method according to claim 1, wherein said effective amount ranges from 0.001 to 100 mg/kg body weight per day.

9. The method according to claim 1, wherein said administering is oral.

10. The method according to claim 1, wherein said administering is non-oral.

11. The method according to claim 1, wherein said administering is enteral.

12. The method according to claim 1, wherein said administering is topical.
